(19) United States
(12) Patent Application Publication
(10) Pub. No.: US 2005/0208553 A1
(43) Pub. Date: Sep. 22, 2005

(54) Methods of detecting and treating microsatellite-instability positive tumors using RIZ

(75) Inventors: Shi Huang, San Diego, CA (US); Robert B. Chadwick, Castro Valley, CA (US)

Correspondence Address: MCDERMOTT, WILL & EMERY, 4370 LA JOLLA VILLAGE DRIVE, SUITE 700, SAN DIEGO, CA, 92122, US

(73) Assignees: The Burnham Institute, La Jolla, CA (US); The Ohio State University, Columbus, OH (US)

(21) Appl. No.: 11/077,465

(22) Filed: Mar. 9, 2005

Related U.S. Application Data
(62) Division of application No. 10/025,450, filed on Dec. 26, 2001, now abandoned.

(51) Int. Cl. A61K 48/00; C12Q 1/68

(52) U.S. Cl. 435/6; 514/44

(57) ABSTRACT

The invention provides a method of inhibiting growth of a microsatellite instability (MSI)-positive tumor. The method is practiced by introducing into an MSI-positive tumor a nucleic acid molecule encoding a RIZ1 polypeptide and expressing the RIZ1 polypeptide in the tumor in an effective amount to inhibit growth of the tumor. Also provided is a method of determining the MSI status of a tumor. The method is practiced by determining in the tumor the number of adenosine (A) nucleotides in a poly(A) tract of a RIZ nucleic acid molecule in the tumor. An abnormal number of adenosine nucleotides in a RIZ poly(A) tract indicates that the tumor is MSI-positive.

This application claims the benefit of U.S. Provisional Application No. 60/256,582, filed Dec. 19, 2000, which is incorporated herein by reference.

This invention was made in part with U.S. Government support under Grant No. RO1-CA76146 awarded by the National Institutes of Health. The U.S. Government has certain rights in this invention.

### BACKGROUND OF THE INVENTION

1. Field of the Invention

The invention relates to the field of cancer and, more specifically, to methods for detecting and treating microsatellite-instability positive (MSI(+)) tumors using the RIZ tumor suppressor gene.

2. Background Information

The retinoblastoma protein (Rb)-interacting zinc finger gene (RIZ) is a candidate tumor suppressor gene belonging to the PR (PRDI-BF1-RIZl homology) or SET (Suvar3-9, Enhancer-of-zeste, Trithorax) domain family of chromosomal regulators involved in chromatin-mediated gene activation and silencing. The PR/SET domain family plays an important role in human cancers as evidenced by genetic alterations of several members of this family. The PR domain of RIZ appears to be a protein-binding interface and can interact with a motif present in the C-terminal region of RIZ.

RIZ gene normally produces two protein products, RIZl and RIZ2, that differ at the N-terminal region by the presence or absence of the PR domain. The RIZl (PR+) product is considered a strong candidate for the tumor suppressor gene present on chromosomal region 1p36, a region commonly deleted in more than a dozen different types of human cancers. RlZl gene expression, but not RIZ2 expression, is commonly silenced in a variety of human tumors and tumor-derived cells, including those of breast, liver, colon, and neuroendocrine tissues (He et al., Cancer Res. 58:4238-4244 (1998); Jiang et al., Int. J. Cancer 83:541-547 (1999)). These tumors were characterized by inactivation of RIZ gene expression, rather than by mutation leading to altered RIZ protein structure. Forced RIZ1 gene expression in such tumor cells has been shown to cause G2/M cell cycle arrest, apoptosis, or both. However, the effect of RIZ1 expression in tumors in vivo has not been determined.

It is now commonly believed that cancers result from the accumulation of genetic alterations in cellular cancer-causing genes. These alterations are thought to be driven by genetic instabilities. Two major genetic instability pathways have been recognized in cancers, chromosomal instability (CIN) and microsatellite instability (MSI). The hallmarks of tumors of the CIN pathway are aneuploidy and loss of heterozygosity. In contrast, tumors of the MSI pathway are usually diploid and show massive instability in simple repeated sequences, or microsatellites.

Microsatellite instability is considered to result from defects in cells' DNA mismatch repair system. This system normally recognizes and restores misincorporated bases or slippage errors that occur during DNA replication. Loss of mismatch repair thus enhances the evolutionary process of mutagenesis and selection which underlies the development of cancer. The mechanism of tumorigenesis of MSI(+) tumors is thought to involve frameshift mutations of microsatellite repeats within coding regions of affected target genes whose inactivation directly contributes to tumor development.

In addition to mutation avoidance, DNA mismatch repair also plays a crucial role in determining the toxicity of a number of DNA-damaging agents that are used in cancer chemotherapy. For example, cell killing by methylating agents, such as N-methyl-N′-nitro-N-nitroguanidine, N-methyl-N-nitrosourea, streptozocin, temozolomide, and dacarbazine; by platinating agents, such as cisplatin and carboplatin; base analog drugs, such as 6-thioguanine; and other chemotherapeutic agents such as busulfan, etoposide and doxorubin, appears to require a functional mismatch repair system. Therefore, administration of chemotherapeutic drugs to patients with MSI(+) tumors may be ineffective.

Gene therapy with tumor suppressor genes is a simpler and less toxic alternative than chemotherapy or radiation. Several clinical trials are underway or proposed in which the tumor suppressor genes p53 or Rb are introduced into tumors which carry mutations in these genes, either using retroviral or adenoviral vectors (see, for example, Roth et al., Oncology 13S5:148-153 (1999). However, MSI(+) tumors generally do not carry mutations in p53 or Rb, and thus gene therapy with these genes is unlikely to be effective. To date, effective gene therapy methods for treating MSI(+) tumors have not been developed.

Accordingly, there exists a need to develop gene therapy approaches to treat MSI(+) tumors, and to develop reliable markers for determining the MSI status of tumors. The present invention satisfies this need and provides related advantages as well.

### SUMMARY OF THE INVENTION

The invention provides a method of inhibiting growth of a microsatellite instability (MSI)-positive tumor. The method is practiced by introducing into an MSI-positive tumor a nucleic acid molecule encoding a RIZ1 polypeptide, and expressing the RIZ1 polypeptide in the tumor in an effective amount to inhibit growth of the tumor.

Also provided is a method of determining the MSI status of a tumor. The method is practiced by determining in the tumor the number of adenosine (A) nucleotides in a poly(A) tract of a RIZ nucleic acid molecule in the tumor. An abnormal number of adenosine nucleotides in the RIZ poly(A) tract indicates that the tumor is MSI-positive.

### BRIEF DESCRIPTION OF THE DRAWINGS

FIG. 1 shows a comparison of the complete human RIZ amino acid sequence (indicated as hRIZ; SEQ ID NO:4) with the complete rat RIZ amino acid sequence (indicated as rRIZ; SEQ ID NO:2). A consensus sequence is shown. Single letter amino acid symbols are used. Amino acids that are identical in hRIZ and rRIZ are shown as a “.”.

FIG. 2 shows the effect of intratumoral or peritumoral injections of either buffer, control Adnull adenovirus, Adp53 adenovirus, or AdRlZl adenovirus on tumor volume of established MSI(+) HCT116 colorectal cell tumors. Arrows indicated the time points when injections were administered.

FIG. 3 shows an immunohistochemical analysis of RIZl expression, p53 expression, and apoptosis (TUNEL) in established HCT116 tumors 2 days after injection of either buffer, control Adnull adenovirus, Adp53 adenovirus, or AdRIZl adenovirus.

### DETAILED DESCRIPTION OF THE INVENTION

The present invention provides a method of inhibiting growth of an MSI-positive tumor, comprising introducing into the tumor a nucleic acid molecule encoding a functional RIZ1 polypeptide, and expressing the polypeptide in the tumor in an effective amount to inhibit growth of the tumor. As disclosed herein, RIZ1 polypeptide is able to inhibit growth of MSI(+) tumor cells in vitro and MSI(+) tumors in vivo. In MSI(+) tumor cells that contain RIZ poly(A) tract frameshift mutations, RIZ1 polypeptide is also able to induce apoptotic cell killing both in vitro and in vivo.

As used herein, the term “microsatellite instability positive tumor cell” or “MSI-positive tumor cell” refers to a tumor cell that exhibits alterations in length of at least 20% of microsatellite marker sequences, in comparison with normal cells from the same individual. Preferably, an MSI-positive (MSI(+)) tumor cell exhibits alterations in at least 40% of microsatellite marker sequences tested, and more preferably at least 60% of microsatellite marker sequences tested, based on determination of alterations in at least five marker sequences. Preferably, an MSI(+) tumor will be determined to contain a frameshift mutation in a poly(A) tract of RIZ. However, due to the unexpectedly high frequency of such mutations in MSI(+) tumors, it is not necessary to predetermine whether RIZ contains mutations to expect to achieve a beneficial effect in a substantial percentage of treated individuals.

Microsatellite sequences are simple-sequence repeats of mono-, di-, tri- or tetra-nucleotides that occur throughout the genome. Panels of microsatellite marker sequences that are considered indicative of MSI status are known in the art (see, for example, Boland et al., Cancer Res. 58:5248-5257 (1998), and primers for analyzing microsatellite sequences are available commercially. Exemplary microsatellite marker sequences include the 5-marker panel of markers consisting of the mononucleotide repeat sequence markers BAT25 (GenBank accession no. 9834508) and BAT26 (GenBank accession no. 9834505); and the dinucleotide repeat sequence markers D5S346 (GenBank accession no. 181171), D2S123 (GenBank accession no. 187953) and D17S250 (GenBank accession no. 177030), proposed in Boland et al., supra (1998). Other suitable microsatellite marker sequences are described, for example, in Boland et al., supra (1998), and Mao et al., Proc. Natl. Acad. Sci. USA 91:9871-9875 (1994).

Microsatellite instability (MSI) is associated with a significant percentage of a variety of sporadic and hereditary tumor types. The methods of the invention are useful in inhibiting growth, and determining MSI status, of both sporadic and hereditary MSI(+) tumors.

In particular, MSI has been found in tumors of the head and neck (e.g. poorly differentiated head and neck tumors), thyroid, esophagus, stomach, colon, prostate (e.g. poorly differentiated prostate tumors), ovary, endometrium (e.g. poorly differentiated endometrial tumors), cervix, breast, melanoma (e.g. metastatic melanoma), small cell lung carcinoma, non small cell lung carcinoma, chronic myelogenous leukemia (e.g. blast crisis), and follicle center cell lymphoma (reviewed in Claij et al., Exp. Cell Res. 246:1-10 (1999)).

The majority of tumors associated with hereditary nonpolyposis colon carcinoma (HNPCC) exhibit microsatellite instability. HNPCC is a familial cancer predisposition syndrome that accounts for about 10% of the total incidence of colorectal cancer. HNPCC is characterized by an early onset of colon cancer, often accompanied by other primary cancers of the colon, endometrium, ovary, small bowel, stomach, urinary tract, sebaceous glands and skin. The methods of the invention are particularly useful in inhibiting growth, and determining MSI status, of HNPCC tumors.

As used herein, the term “tumor” refers to a localized growth of cancer cells, which can be the site where a cancer originally formed or can be a metastatic lesion. The term “tumor cell” refers to a malignant cell, either within a tumor or metastatic lesion, or isolated from a tumor or metastatic lesion. A tumor cell isolated from a tumor or metastatic lesion can optionally be cultured for one or several generations.

As used herein, the term “inhibits growth of a tumor,” refers to any slowing of the rate of tumor cell proliferation, arrest of tumor cell proliferation, or killing of cells within the tumor, such that the rate of tumor growth is reduced in comparison with the observed or predicted rate of growth of an untreated control tumor. The term “inhibits growth” can also refer to a reduction in size or disappearance of the tumor, as well as to a reduction in its metastatic potential. Those skilled in the art can readily determine, by any of a variety of suitable indicia, whether tumor growth is inhibited.

Inhibition of tumor growth can be evidenced, for example, by arrest of tumor cells in a particular phase of the cell cycle. For example, as described in Example I, below, exogenous RIZ1 expression causes arrest at the G2/M phase of the cell cycle in MSI(+) tumor cells, whether or not they contain RIZ poly(A) tract frameshift mutations.

Inhibition of tumor growth can also be evidenced by direct or indirect measurement of tumor size. For example, as described in Example III, below, exogenous RIZ1 expression causes a reduction in the rate of increase in volume of MSI(+) tumors. In human cancer patients, such measurements generally are made using well known imaging methods such as magnetic resonance imaging, computerized axial tomography and X-rays. Tumor cell growth can also be determined indirectly, such as by determining the levels of circulating carcinoembryonic antigen (CEA), prostate specific antigen or other tumor-specific antigens that are correlated with tumor growth. Inhibition of tumor growth is also generally correlated with prolonged survival and/or increased health and well-being of the subject.

As described in Examples I and III, below, expression of exogenous RIZ1 in MSI(+) tumor cell lines and tumors, particularly MSI(+) tumors with RIZ poly(A) tract frameshift mutations, also can induce apoptosis. As used herein, the term “induces apoptosis” refers to the promotion of a form of programmed cell death characterized by DNA fragmentation. Apoptosis can be determined by methods known in the art. For example, as described in. Example III, below, kits are commercially available that detect the presence of fragmented DNA by in situ immunohistochemistry (e.g. Apoptag, available from Intergen, Purchase, N.Y.). Additionally, as described in Example I, below, apoptosis can also be determined by FACS analysis, in which apoptotic cells exhibit a sub-G1 DNA content, indicating DNA fragmentation.

The invention method is practiced by introducing into an MSI(+) tumor a nucleic acid molecule encoding a RIZ1 polypeptide, and expressing RIZ1 in an effective amount in the tumor. As used herein, the term “RIZ1 polypeptide” refers to a polypeptide having the human RIZ1 amino acid sequence designated SEQ ID NO:4, or to a functional fragment thereof. The term “RIZ1 polypeptide” also refers to a polypeptide having one or more minor modifications to the sequence designated SEQ ID NO:4, so long as the polypeptide retains the ability to inhibit growth of an MSI(+) tumor or tumor cell. Minor modifications include one or more additions, deletions or substitutions to the sequence of SEQ ID NO:4, that do not deleteriously affect its growth-inhibitory ability.

The cloning of the human RIZ gene designated SEQ ID NO:3, which encodes a RIZ1 polypeptide having the amino acid sequence designated SEQ ID NO:4, as well as the cloning of the rat RIZ gene designated SEQ ID NO:1 (which encodes SEQ ID NO:2), and the mouse RIZ gene designated SEQ ID NO:8 are described in U.S. Pat. Nos. 6,069,231, 5,831,008 and 5,811,304, and in Buyse et al., Proc. Natl. Acad. Sci. USA 92:4467-4471 (1995).

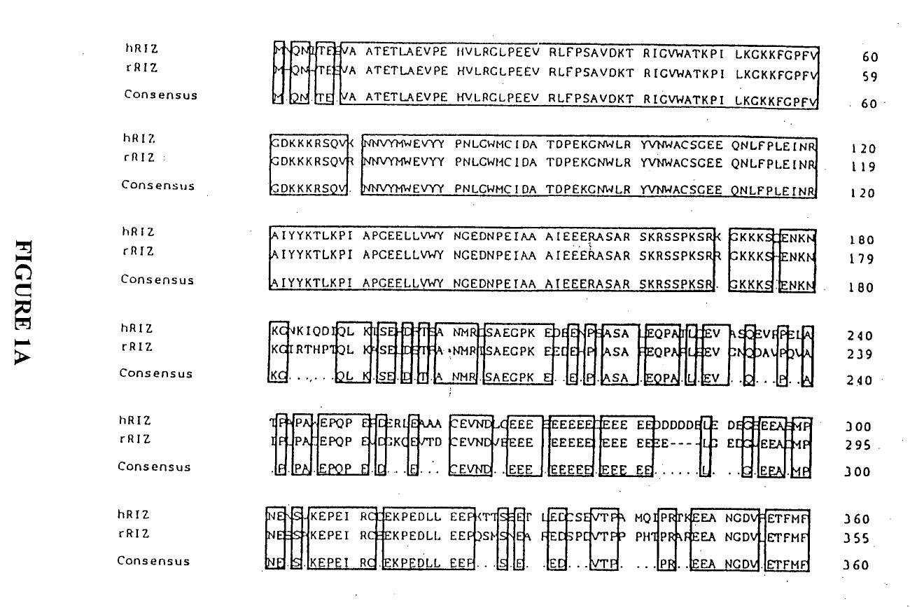

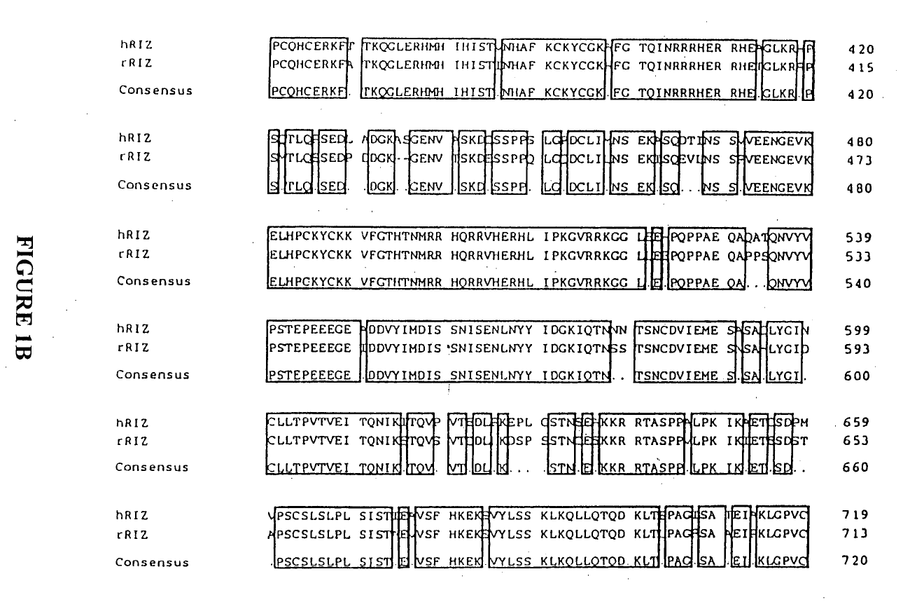

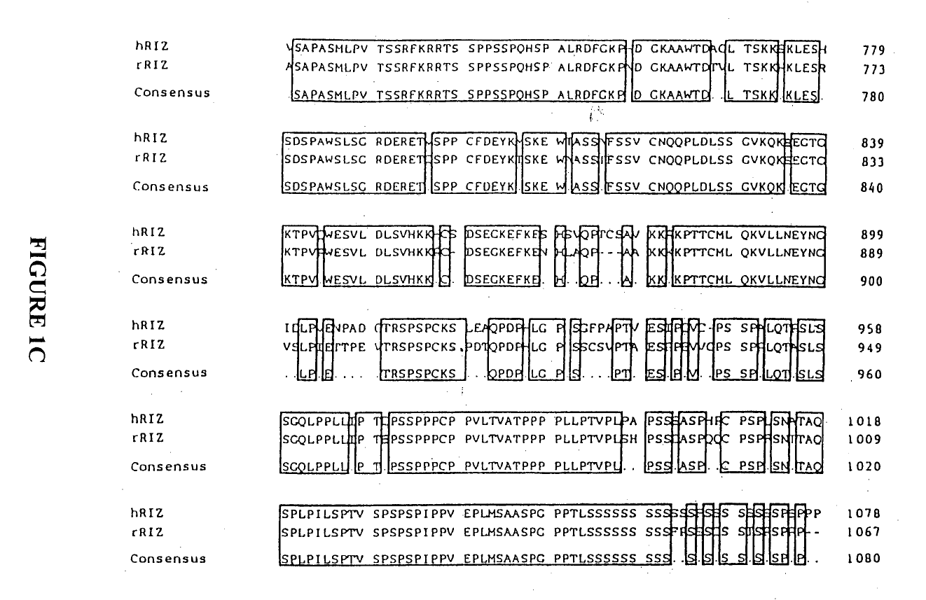

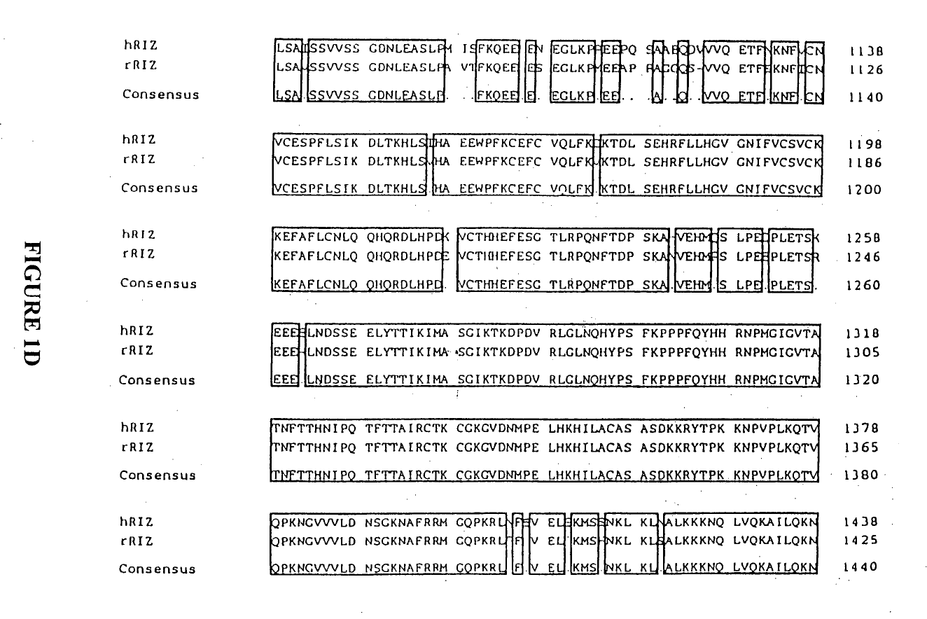

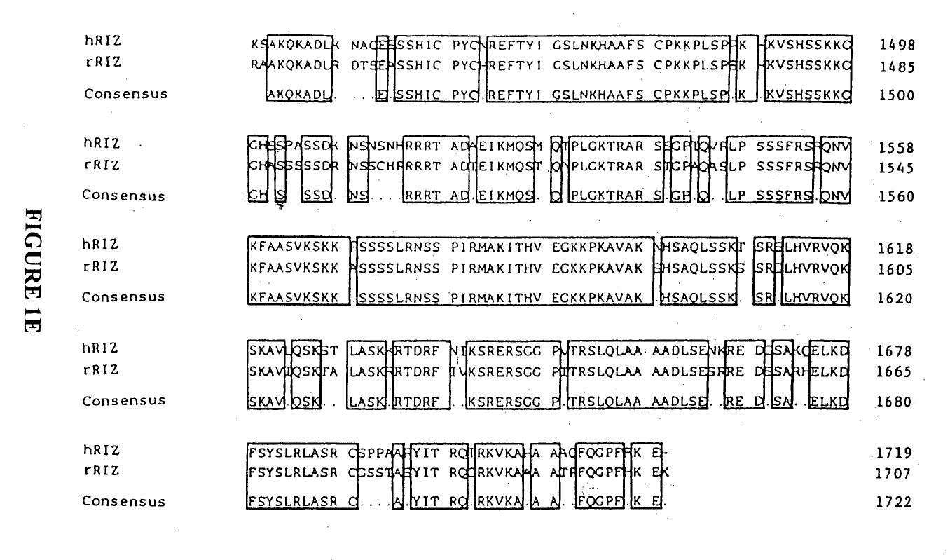

A RIZ1 polypeptide can have at least 70% identity to the amino acid sequence designated SEQ ID NO:4. Preferably, a RIZ1 polypeptide will have at least 75% identity, including at least 80%, 85%, 90%, 95%, 98%, 99% or greater identity to SEQ ID NO:4. For example, a RIZ1 polypeptide can be a RIZ1 from another mammalian species. As shown in FIG. 1, human RIZ1 (SEQ ID NO:4) and rat RIZ1 (SEQ ID NO:2) are 84% identical over their sequences.

It is well known in the art that evolutionarily conserved amino acid residues and domains are more likely to be important for maintaining biological activity than less well-conserved residues and domains. Thus, it would be expected that substituting a residue that is highly conserved among RIZ polypeptides across species, with a non-conserved residue may be deleterious, whereas making the same substitution at a residue which varies widely among species would likely not have a significant effect on biological activity.

Substitutions to the amino acid sequence designated SEQ ID NO:2 can either be conservative or non-conservative. Conservative amino acid substitutions include, but are not limited to, substitution of an apolar amino acid with another apolar amino acid (such as replacement of leucine with an isoleucine, valine, alanine, proline, tryptophan, phenylalanine or methionine); substitution of a charged amino acid with a similarly charged amino acid (such as replacement of a glutamic acid with an aspartic acid, or replacement of an arginine with a lysine or histidine); substitution of an uncharged polar amino acid with another uncharged polar amino acid (such as replacement of a serine with a glycine, threonine, tyrosine, cysteine, asparagine or glutamine); or substitution of a residue with a different functional group with a residue of similar size and shape (such as replacement of a serine with an alanine; an arginine with a methionine; or a tyrosine with a phenylalanine).

Additions to a RIZ polypeptide include, but are not limited to, the addition of “tag” sequences at the N or C termini, or between domains. Such tag sequence include, for example, epitope tags, histidine tags, glutathione-S-transferase (GST), and the like, or sorting sequences. Such additional sequences can be used, for example, to facilitate expression or identification of a recombinant RIZ1.

Exemplary modifications to the RIZ1 polypeptide sequence designated SEQ ID NO:4 include normal allelic variants of SEQ ID NO:4, such as RIZ1 in which the first three amino acids by the amino acid sequence designated SEQ ID NOS:6 or 7. Another exemplary allelic variant contains a single nucleotide change of T₉₆₉ to A₉₆₉, leading to a change of amino acid residue D₂₈₃ to E₂₈₃. The T₉₆₉ allele is estimated to be two times more frequent than the A₉₆₉ allele. A further allelic variant of SEQ ID NO:4 is a variant in which the proline at residue 704 is deleted.

In one embodiment, a RIZ1 polypeptide expressed in a tumor in a method of the invention comprises the PR domain of SEQ ID NO:4, designated SEQ ID NO:5, or a sequence at least 95% identical thereto, such as the PR domain of rat RIZ.

RIZ1 contains a region of about 100 residues near the N-terminus that is designated the “PR domain” because it is 42% homologous with a similar N-terminal region from PRDI-BF1 and Blimp-1. The PR domain is also homologous to an N-terminal portion of the mammalian Evi-1 protein (Morishita et al., Cell 54:831-840 (1988); Morishita et al. Oncogene 5:936-971 (1990)) and to an N-terminal portion of the C. elegans egl-43 protein, which is a homolog of Evi-1 (Garriga et al., Genes Devel. 7:2097-2109 (1993)).

The PR domain has been demonstrated to be required for the negative regulatory function of RIZ (He et al., Cancer Res. 58:4238-4244 (1998)), and may be involved in chromatin-mediated transcriptional activation or repression (Xie et al., J. Biol. Chem. 272:26360-26366 (1997); Huang et al., J. Biol. Chem. 273:15933-15939 (1998)).

In general, a PR domain is about 100 to about 120 amino acids in length and contains three highly conserved sequences, designated blocks A, B and C, which consist of about 10 to about 12 amino acids, separated by less conserved sequences of about 20 to about 35 amino acids. Each of blocks A, B and C is encoded by an individual exon. The PR domains of rat RIZ (a.a. positions 36 to 151 of SEQ ID NO:2) and human RIZ (a.a. positions 37 to 152 of SEQ ID NO:4; designated SEQ ID NO:5) are identical except that the human RIZ contains a lysine at a.a. position 70, whereas the rat RIZ contains an arginine at the equivalent position (a.a. position 69). Additionally, analysis of a cDNA encoding a portion of the mouse RIZ protein that includes blocks B and C of a PR domain peptide (SEQ ID NOS:8 and 9) revealed that the deduced amino acid sequence (i.e. the first 75 amino acids of SEQ ID NO:9) is identical to the corresponding region of the PR domain in human RIZ (i.e. amino acids designated as positions 42-116 of human RIZ SEQ ID NO:5).

The RIZ1 polypeptide includes several other motifs of interest, including an RB-binding motif related to that of the E1A oncoprotein; eight zinc finger motifs; and a C-terminal PR-binding motif. The interaction of the N-terminal PR domain with the C-terminal PR binding domain may be necessary for homo- or hetero-oligomerization of RIZ, and for interactions with other proteins.

Those skilled in the art can readily determine, by the methods disclosed herein, whether a RIZ1 polypeptide that is a modification or fragment of SEQ ID NO:4 retains the ability to inhibit growth of an MSI(+) tumor or tumor cell. As disclosed in Examples I and III, the ability of a RIZ1 polypeptide to inhibit MSI(+) tumor cell growth in vitro is predictive of its ability to inhibit MSI(+) tumor growth in vivo. Therefore, a nucleic acid molecule encoding a RIZ1 polypeptide that is a modification or fragment of SEQ ID NO:4 can be introduced either into an MSI(+) tumor, or into a tumor cell, such as an HCT116+ tumor cell line, to determine whether it retains the ability to inhibit growth. If so, the nucleic acid molecule is suitable for use in the methods of the invention.

A nucleic acid molecule encoding a RIZ1 polypeptide can be contained in any suitable vector for expression in a mammalian subject. Appropriate vectors include, but are not limited to, viral vectors such as retroviral vectors (e.g. replication-defective MuLV, HTLV, and HIV vectors); adenoviral vectors; adeno-associated viral vectors; herpes simplex viral vectors; and non-viral vectors (e.g. viral genomes, plasmids and phagemids) (see, for example, Kaplitt and Loewy, Viral Vectors: Gene Therapy and Neuroscience Applications Academic Press, San Diego, Calif. (1995); Chang, Somatic Gene Therapy CRC Press, Boca Raton, Fla. (1995)). Methods of cloning nucleic acid molecules encoding any desired sequences are well known in the art.

Adenoviral vectors are particularly advantageous in that they can transduce both relicating and non-replicating cells, and can be grown to high titers in vitro. Additionally, adenoviral vectors do not integrate into the host genome, resulting in a safety advantage. Adenoviral vectors have been successfully used to introduce the p53 tumor suppressor gene into tumors, with low toxicity, high levels of gene expression, and therapeutic efficacy (e.g. Swisher et al., J. Natl. Cancer Inst. 91:763-771 (1999); Clayman, Seminars in Oncology 27S8:39-43 (2000)).

Optionally, a viral vector or other vector can be constructed to express a nucleic acid encoding a RIZ in a tissue specific manner by incorporating a tissue-specific promoter or enhancer into the vector (e.g. Dai et al., Proc. Natl. Acad. Sci. USA 89:10892-10895 (1992)). Alternatively, a nucleic acid molecule encoding a RIZ1 polypeptide need not be incorporated into a vector, so long as it is linked to appropriate nucleotide sequences required for transcription and translation.

Appropriate formulations for delivery of nucleic acid molecules can be determined by those skilled in the art, depending, for example, on the type of vector (e.g. infectious or non-infectious) and route of delivery. Suitable formulations include, for example, incorporating the nucleic acid molecules into liposomes; mixing the nucleic acid molecules with polycationic agents; and conjugating the nucleic acid molecules to targeting molecules (e.g. antibodies, ligands, lectins, fusogenic peptides, or HIV tat peptide). Gene therapy methods, including considerations for choice of appropriate vectors, promoters and formulations, are reviewed, for example, in Anderson, Nature 392:25-30 (1998).

A nucleic acid molecule encoding a RIZ1 polypeptide can be administered to a subject by various routes such that the polypeptide is expressed in an effective amount in the tumor. In a preferred embodiment, administration of the nucleic acid molecule is local, such as intra- or peri-tumoral, which can be achieved, for example, by injection or particle bombardment. Depending on the tumor location, local administration can optionally be performed in conjunction with a surgical procedure, or by using imaging procedures to direct a delivery instrument to the tumor site (e.g. Swisher et al., supra (1999)). Local administration can be advantageous is that there is no dilution effect and, therefore, the likelihood that a majority of the tumor cells will be contacted with the nucleic acid molecule is increased.

Administration of a nucleic acid molecule encoding RIZ1 can alternatively be systemic, such as via intravenous or intra-arterial injection, or via administration into a body compartment (e.g. intraperitoneal or intracerebral compartments). Systemic routes are particularly advantageous for treating disseminated tumors.

Receptor-mediated DNA delivery approaches can be advantageous when administering a nucleic acid molecule encoding a RIZ1 polypeptide at a site other than at the tumor site. For example, a viral particle can be complexed with a tissue-specific or tumor-specific ligand or antibody via a bridging molecule. Following administration, the viral particles will circulate until they recognize host cells with the appropriate target specificity for infection.

The invention also provides a method of determining MSI status of a tumor, comprising determining the number of adenosine (A) nucleotides in a poly(A) tract of a RIZ nucleic acid molecule in the tumor, wherein an abnormal number of adenosine nucleotides in the RIZ poly(A) tract indicates that the tumor is MSI-positive.

As disclosed herein, frameshift mutations in either of two poly-adenosine tracts of the RIZ gene were detected in a high percentage of MSI(+) tumor cells and cell lines, including tumors and cell lines derived from colon, gastric and endometrial tissue. In contrast, RIZ poly(A)-tract mutations were not detected in MSI-tumor cells and cell lines. Accordingly, the determination that a tumor contains a RIZ poly(A) tract frameshift mutation strongly predicts that the tumor is MSI(+).

Scanning of RIZl cDNA sequence revealed two potentially hypermutable polyadenosine tracts within its coding region in exon 8: one (A)₈ tract at residues 4393-4400 of SEQ ID NO:3, and one (A)₉ tract at residues 4582-4590 of SEQ ID NO:3. The (A)₈ tract of the RIZ gene is immediately 5′ of the most C-terminal zinc finger domain, and frameshift mutation in this tract predicts termination of translation such that this zinc finger would be truncated. The (A)₉ tract is 30 nt (10aa) past this same zinc finger domain. Frameshift mutations in either tract, caused by an addition or deletion of an A residue, are predicted to lead to loss of the C-terminal domain of the RIZ protein that is involved in PR binding.

Methods of determining the number of A residues in a RIZ gene (A)₈ tract or (A)₉ tract are known in the art and include, for example, PCR amplification of RIZ genomic DNA, or RIZ cDNA, followed by SSCP analysis and/or direct sequencing of the region of interest. Exemplary methods of determining the number of A residues in a RIZ gene (A)₈ tract or (A)₉ tract are described in Examples I and II, below.

The DNA sample to be analyzed is preferably obtained directly from the tumor, such as by a tissue biopsy. Either fresh or fixed tissue sections can be assayed. For certain applications of the method, it may be preferable to analyze tumor cells released into the urine, blood or lymph, or tumor cells that have been passaged in culture.

As described previously, tumors that are MSI(+) may be less susceptible to certain types of chemotherapy than tumors that are MSI-. Therefore, it is important to be able to predict the MSI status of a tumor prior to initiating treatment, such that therapy can be optimized. As described herein, tumors that are MSI(+) are susceptible to growth inhibition and apoptosis by introduction and expression of exogenous RIZ1. Accordingly, RIZ gene therapy can be used as an alternative to chemotherapy, or in combination with other therapies.

The following examples are intended to illustrate but not limit the present invention.

### EXAMPLE I

### RIZ Mutations in MSI(+) Tumors, and Effect of Ectopic Expression of RIZ in MSI(+) Tumors

This example shows that RIZ poly(A)-tract frameshift mutations are present in a high percentage of MSI(+) tumor cells of a variety of different cell types. This example also shows that expression of a nucleic acid molecule encoding functional RIZ in an MSI(+) tumor cell containing a RIZ-poly(A)-tract frameshift mutation induces cell cycle arrest and apoptosis of the tumor cells.

Materials and Methods

Tissue Samples and Cell Lines. 22 MSI(−) tumors were studied. 8 of these were selected because they previously had been found to display a CIN (chromosomal instability) phenotype, including LOH at two closely linked markers, D1S228 [32.4 centimorgans (cM)] and D1S507 (36.2 cM) (Canzian et al., Cancer Res. 56:3331-3337 (1996)). In 14 tumors, the LOH status at 1p was unknown. Additionally, 3 MSI(−) lines were obtained from the American Type Culture Collection (ATCC). The MSI(−) lines were MDAMB231, MDAMB435S, and SKBR3 (breast cancer), which had been previously characterized for RIZ expression (He et al., Cancer Res. 58:4238-4244 (1998)).

In addition, 24 MSI(+) tumors from hereditary nonpolyposis colorectal cancer (HNPCC) patients were studied (Aaltonen et al., N. Eng. J. Med. 338:1481-1487 (1998)). Moreover, 11 MSI(+) were obtained from the ATCC. The MSI(+) lines were DLD1, LS411N, SW48, HCT116, LoVo, HCT15, and LS174T (colorectal cancer); MDAH2774 and SK-OV3 (ovarian cancer); AN3CA (endometrial cancer); and DU145 (prostate cancer).

Loss of heterozygosity (LOH) analysis. Primary normal/tumor pairs were investigated by using fluorescently labeled microsatellites. Primer sequences were obtained from the Genome Database (http://gdbwww.gdb.org/). Amplifications of each microsatellite were done in 15 μl volumes with 10 ng of each respective genomic DNA, 8 pmol of each primer (5′ primer, fluorescently labeled), 100 μM each dNTP, 0.6 unit of AmpliTaq Gold DNA Polymerase (PE Biosystems, Foster City, Calif.), 10 mM Tris.HCl (pH 8.3), 50 mM KCl, and 2 mM MgCl2. PCR products were loaded onto a 377XL sequencer (PE Biosystems). Allele size and fluorescent intensity were determined by GENESCAN and GENOTYPER software (PE Biosystems). LOH was analyzed by determining the fluorescent intensity of each allele and calculating the ratio (Canzian et al., Cancer Res. 56:3331-3337 (1996)). A sample was scored as showing LOH if an allelic ratio of <0.67 or >1.5 was obtained.

Because of a high degree of MSI observed in the HNPCC tumor DNAs, single-nucleotide polymorphisms (SNPs) were also used to determine LOH in the subset of 24 HNPCC normal/tumor DNA pairs. Primer sequences were obtained from the Human SNP database (http://www-genome.wi.mit.edu/SNP/human/index.html). SNPs were amplified in 25 μl volumes with 100 nmol of each of the respective PCR primers, 25 ng of genomic DNA, 100 μM each dNTP, 1.0 unit of AmpliTaq Gold DNA Polymerase (Perkin-Elmer), 10 mM Tris.HCl (pH 8.3), 50 mM KCl, and 2 mM MgCl₂. PCR products were purified by using exonuclease 1 and shrimp alkaline phosphatase (Amersham Life Sciences) and directly sequenced in one direction with one of the amplification primers and the BigDye Terminator chemistry (PE Biosystems). Samples that failed or sequenced poorly were resequenced in the other direction with the other amplification primer. LOH determination was done by a method similar to the microsatellite analysis.

Mutation Analysis. Candidate genes were screened for mutations by direct sequencing of genomic PCR products: To facilitate direct sequencing of PCR products, all primers were tailed with M13-forward (TGTAAAACGACGGCCAGT; SEQ ID NO:10) and M13-reverse (CAGGAAACAGCTATGACC; SEQ ID NO:11) sequences. PCRs were performed in 25-μl volumes with 100 nmol of each of the respective PCR primers, 25 ng of genomic DNA, 100 μM each dNTP, 1.0 unit of Taq Gold DNA polymerase (Perkin-Elmer), 10 mM Tris-HCl (pH 8.3), 50 mM KCl, and 2 mM MgCl2. PCR fragments were purified by using the Exonuclease I/Shrimp Alkaline Phosphatase PCR Product Presequencing Kit (United States Biochemical). After purification according to the manufacturer's protocol, 2 μl of the PCR products were sequenced by using the BigDye Terminator AmpliTaq FS Cycle Sequencing Kit (PE Biosystems).

Expression Analysis. RNA for expression analysis was isolated by using the RNAeasy Mini Kit (Qiagen, Chatsworth, Calif.). Reverse transcription of isolated RNA was done by using Superscript RT (Life Technologies, Rockville, Md.), and cDNA amplification was done by using the GeneAmp Gold RNA PCR kit (PE Biosystems). Primers used for determination of RIZ1- and RIZ2-specific PCRs were as described (He et al., supra (1998)). Glyceraldehyde-3-phosphate dehydrogenase (GAPDH)-specific primers were used as a control (Leygue et al., Cancer Res. 58:1348-1352 (1998)).

Immunoprecipitation and Immunoblot Analysis. Immunoprecipitation and immunoblot analysis was performed according to procedures described in Buyse et al., Proc. Natl. Acad. Sci. USA 92:4467-4471 (1995). Cell extracts were immunoprecipitated with RIZ antiserum 1637 against the N terminus of RIZ2, or preimmune serum. Immunoprecipitated products were resolved on a 5% SDS gel followed by immunoblot analysis using mouse antiserum KGSE against the N terminus of RIZ2.

Analysis of Cells Expressing Ectopically Introduced RIZ1. Analysis of cells expressing ectopically introduced RIZ1 was performed as described in He et al., supra (1998). Briefly, colon cancer cells were seeded at 2×10⁵ cells in 6-cm dishes and infected with recombinant adenovirus at a multiplicity of infection of 100. At 48 hr postinfection, cells were processed for DNA histogram analysis.

Results

Deletion Mapping of 1p. Most of the sporadic colorectal cancer tumors showed deletion of the entire 1ptel region up to 40 cM, where there was a return to heterozygosity in several tumors (see FIG. 1 of Chadwick et al., Proc. Natl. Acad. Sci. USA 97:2662-2667 (2000)). The region of return to heterozygosity is telomeric to several candidate tumor suppressor genes and oncogenes, including PAX7 at 46.2 cM, PLA2 at 46.2 cM, E2F2 at 52.4 cM, and MYCL at 71 cM. In comparison with MSI(−) tumors, the area of shared deletion was more restricted in MSI(+) tumors from HNPCC patients. Although the majority of the SNP markers closest to 1ptel were not informative, a region of common deletion was identified by the marker WIAF-481 at 32.2 cM, which showed LOH in 8 of 15 (53%) informative HNPCC tumors. The allelic imbalance values for SNPs were highly reproducible, indicating the validity of these markers in the deletion mapping of MSI(+) tumors in particular. In comparison, a much lower rate of LOH was observed for nearby markers such as D1S450 (1/11) at 22.9 cM and D1S228 (1/8) at 32.4 cM.

Identification of RIZ as a Candidate Gene for 1p Alterations. Functional candidate genes in the region of 32.2 cM from 1ptel were examined and screened for mutations. The RIZ gene maps to 32.2 cM on GeneMap 99 (http://www.ncbi.nlm.nih.gov/genemap99/). RIZ is within 370 kb of D1S228, as inferred from yeast artificial chromosome analysis (Leygue et al., supra (1998)). RIZ lies in 3′ to 5′ orientation from the telomere of chromosome 1p. In HNPCC tumors, there was a gradual decline of the LOH rate from WIAF-481 to RIZ 3′ to RIZ 5′. Three intragenic polymorphisms of RIZ were analyzed for LOH, including a codon Pro-704 deletion at exon 8, an intron 4 CA repeat, and a SNP flanking exon 4 (GAT to GAC 18 bases 3′ of the coding exon 4 sequence). The LOH rates for these RIZ markers were 21% (3/14), 9% (1/11), and 0% (0/8), respectively. Tumor 5393T showed LOH of exon 8 Pro-704 but retention of the other intragenic markers, consistent with location of a deletion break point inside the RIZ gene. Analysis of MSI(−) tumors that were not preselected for 1p LOH revealed LOH rates of 30% (4/13), 23% (3/13), and 60% (3/5), respectively. Tumor 7T-OSU showed LOH of RIZP704 but retention of heterozygosity of RIZ intron 4 CA, again indicating a deletion breakpoint within RIZ. The location of RIZ in the vicinity of the common LOH region and the observed deletion breakpoints within RIZ suggest that RIZ is a candidate target of 1p36 alterations in both hereditary and sporadic colorectal cancers.

Examination of the coding sequence of RIZ showed potentially hypermutable tracts of (A)₈ and (A)₉ in exon 8 of the RIZ gene. In 9 of 24 (37.5%) of the MSI(+) HNPCC tumors, frameshift mutations were found in either the (A)₈ [one MSI(+) tumor] or the (A)₉ [eight MSI(+) tumors] tract. Furthermore, 6 of 11 MSI(+) cell lines (HCT116, LoVo, LS411N, LS174T, MDAH2774, and AN3CA) showed frameshift mutations in the (A)₉ tract. In cell lines HCT116 and AN3CA, the mutation was homozygous/hemizygous, resulting in biallelic involvement. None of the 23 tested MSI(−) sporadic colorectal cancers contained mutations in either of the polyadenosine tracts, indicating that these regions were mutational hotspots in MSI(+) tumors only.

RIZ Frameshift Mutations Are Clonally Selected in Tumorigenesis. To investigate whether similar mononucleotide tracts in other genes are involved to a comparable degree, the coding (A)₈ tracts of the PMS2 and DNA polymerase genes were analyzed. Frameshift mutations in these tracts were completely absent in the MSI(+) tumors (P =0.0005). MSI(+) tumors were further screened for mutations in the (A)₉ tracts of the RECQL, BLM, and KIAA0355 genes. One of 24 MSI(+) tumors had 1-bp deletions in the RECQL and KIAA0355 genes (P=0.005). MSI(+) tumors were screened further for mutations in the (A)₉ tracts of the RECQL, BLM, and KIA0355 genes. Of 24 MSI(+) tumors, 1 had 1-bp deletions in the BLM gene (P=0.005). Taken together, these results suggest that the RIZ frameshift mutations were specifically selected during the clonal evolution of colorectal tumorigenesis.

Decreased RIZ1 Expression in Cell Lines. Previous reports have shown that RIZ is expressed in two alternative transcripts, RIZ1 and RIZ2 (He et al., supra (1998)). RIZ1 is commonly lost, whereas RIZ2 is present uniformly in several 1p36-linked cancer types (He et al., supra (1998); Jiang et al., Int. J. Cancer 83:541-547 (1999); Jiang et al., Histol. Histopathol. 15:109-117 (2000)). To determine whether expression was affected in RIZ-mutated colorectal tumors, expression by the reverse transcription-PCR was examined as described in He et al., supra (1998); Jiang et al., supra (1999); and Jiang et al., supra (2000). Because of total overlap between the smaller RIZ2 and the larger RIZ1 transcripts, transcription was not measured for RIZ2 alone; hence, one reaction is specific for RIZ1, whereas the other measures RIZ1+RIZ2. Breast cancer cell line MDAMB435S was used as a control that expresses only the RIZ2 isoform, and MDAMB231 was used as a control cell line that expresses both RIZ1 and RIZ2 isoforms (He et al., supra (1998)).

MSI(+) colorectal cancer cell lines (4 of 11) showed reduced or lost mRNA expression of RIZ1 in the presence of abundant RIZ1+RIZ2 transcript, supporting observations that an imbalance in the amounts of RIZ1 and RIZ2 is associated significantly with malignancy (He et al., supra (1998); Jiang et al., supra (1999); and Jiang et al., supra (2000)). In three cell lines with altered RIZ1 expression, frameshift mutations in the polyadenosine tracts were present, including cell line HCT116, which is homozygous or hemizygous for this mutation. Of these cell lines, LS411N was found to express only the RIZ2 isoform, whereas LoVo showed reduced expression of RIZ1. Also, SW48 showed reduced or absent expression of RIZ1, but had no mutations in the adenosine repeats of RIZ. The RIZ sequence is large (nearly 8 kb), and it is possible that there are other areas in the gene or in the promoter region that could be affected, resulting in altered RIZ expression in these cell lines.

RNA isolated from frozen tissue from the mutation-positive HNPCC tumors did not show clear expression changes of RIZ. This finding is likely caused by the contamination of normal tissue in the isolated RNA tumor samples. Titration experiments of cell-line RNA showed that even with a mixture of 90% RIZ2-expressing mRNA to 10% RIZ1- and RIZ2-expressing mRNA, the RIZ1 and RIZ2 isoform is PCR amplified.

Frameshift Mutation Leads to Expression of Truncated RIZ Proteins. The frameshift caused by the deletion of one adenosine at the (A)₉ tract (at nucleotide position 4700 of the RIZ1 coding sequence) is expected to cause the fusion of truncated RIZ1 and RIZ2 lacking the C-terminal 219 aa with a novel reading frame of 76 aa. This would lead to the expression of mutant RIZ1 and RIZ2 that are 157 residues shorter than their wild-type counterparts. To confirm that such truncated RIZ1 and RIZ2 proteins indeed were expressed from the mutant allele, immunoprecipitation and immunoblot analysis was performed using the HCT116 cell line that is homozygous or hemizygous for the frameshift mutation. The usual pattern of RIZ protein expression in DLD1 cells that expressed both RIZ1 and RIZ2 mRNAs was first confirmed. In all tumor cell lines studied previously, RIZ1 protein was at low levels and difficult to detect, whereas RIZ2 protein was at higher levels and readily detectable (He et al., supra (1998); Buyse et al. supra (1995); Liu et al., J. Biol. Chem. 272:2984-2991 (1997)). Similarly, in DLD1 cells, RIZ2 protein of 250 kDa was detected, and RIZ1 protein was at low or undetectable levels. In contrast, full-length RIZ2 protein was not detected in the HCT116 cell line, but instead a shorter protein of 230 kDa was observed, consistent with truncation of RIZ2 protein by the frameshift mutation. Although the experiment was not informative for RIZ1 protein, a truncated RIZ1 protein could be inferred from the results on RIZ2.

RIZ1 Causes G2/M Arrest, Apoptosis, or Both in Colorectal Cancer Cell Lines. Adenovirus-mediated RIZ1 expression has been shown to cause G2/M arrest, apoptosis, or both in breast and liver cancer cell lines, which were not MSI(+). The effects of adenovirus-mediated RIZ1 expression on HCT116 and DLD1 colon cancer cell lines were examined. Immunoblot analysis confirmed full-length RIZ1 protein expression in both cell lines upon infection with AdRIZ1 at a multiplicity of infection of 100. The fraction of infected cells in G2/M increased significantly at 48 h after AdRIZ1 infection in HCT116 and DLD1 cells. At 48 and 72 h postinfection with AdRIZ1, HCT116 cells showed sub-G1 DNA content indicating apoptotic cell death (Table 1). However, few cells with sub-G1 DNA content were observed in DLD1. The results show that RIZ1 caused G2/M arrest and apoptosis in HCT116 cells, but only G2/M arrest in DLD1 cells.

TABLE 1

| Cell Line | Apoptosis | G1 | S | G2/M |
|---|---|---|---|---|
| HCT116 | +25 ± 2.0 | −47 ± 1.3 | +6.1 ± 0.7 | +20 ± 0.8 |
| DLD1 | +9.5 ± 1.2 | −28 ± 2.1 | −2.2 ± 0.5 | +26 ± 1.7 |

In Table 1, DNA histogram analysis was performed 72h after adenovirus (Ad) RIZ1 or AdNull virus infection. The values shown represent the difference in percentage of cell populations between AdRIZ1- and AdNull-infected cells. The increase caused by AdRIZ1 over AdNull is indicated by a plus sign (+) and a decrease is indicated by a minus sign (−). The values represent the mean±SD of three experiments.

The results described above indicate that the function of RIZ in tumors may be impaired by somatic events in at least two different ways. In MSI(+) tumors, frameshift mutations in the 3′ end of the gene interfere with the interaction between the C terminus of the protein and its N-terminal PR domain. In MSI(−) tumors (CIN pathway), mutations or deletions of the PR domain of RIZ1 may have similar effects. In the series of tumors examined, RIZ was affected by either LOH or frameshift mutation, but not both, suggesting that LOH and frameshift mutations have similar, alternative functions in RIZ-associated tumorigenesis.

### EXAMPLE II

### RIZ Poly(A)-Tract Frameshift Mutations in MSI(+) Gastric Cancers

This example shows that RIZ poly(A)-tract frameshift mutations occur with high frequency in MSI(+) gastric cancers.

To examine the role of RIZ in MSI(+) tumors, a total of 179 primary gastrointestinal and endometrial tumors from patients undergoing surgery were analyzed. Among them, 109 tumors were characterized as MSI-High, including 40 gastric carcinomas (K), 18 endometrial cancers (E or AN), and 51 colorectal cancers (AC, IC or AS). MSI-High status in primary tumors was defined according to the criteria proposed by Boland et al., supra (1998). The source of tumor samples is described in Kong et al., Nat. Genet. 17:143-144 (1997); Yamamoto et al., Cancer Res. 57:4420-4426 (.1997); and Kim et al., Lab. Investig. 79:1113-1120 (1999). A panel of MSI(+) cell lines derived from colon (HCTl16, SW-48, LOVO, LS44lN, LSl80, LSl74T, DLDl, HCTl5, HCT8), prostate (DUl45), breast (Cal-51), and uterus (AN3CA, SK-UT-1B) cancers was also screened (obtained from ATCC). The MSI(−) colon cancer cell line SW620 was also included as a control.

Frameshift mutations at the (A)₈ and (A)₉ tracts in RIZ were detected by PCR with Vent DNA polymerase and SSCP analysis. The (A)₈ tract was amplified by PCR with primers RIZA8-F, 5′-GAGCTCAGCAAAATGTCGTC-3′ (SEQ ID NO:12) and RIZA8-R, 5′-CAAGTCGGCCTTCTGCTTTG-3′ (SEQ ID NO:13). The (A)₉ tract was amplified by PCR with primers RIZA9-F, 5′-TCTCACATCTGCCCTTACTG-3′ (SEQ ID NO:14) and RIZA9-R, 5′-GTGATGAGTGTCCACCTTTC-3′ (SEQ ID NO:15). PCR was carried out as described in Yamamoto et al., supra (1997). PCR was performed with primers RIZA8-F and RIZA9-R for SSCP analysis, as described in Yamamoto et al., supra (1997). The mutated bands in the SSCP gel were sequences using the Big Dye terminator cycle sequencing kit (Perkin-Elmer Corp.).

RIZ mutations were detected in 19 of 40 (48%) MSI(+) gastric carcinomas, 6 of 18 endometrial cancers (33%), 14 of 51 (26%) colorectal cancers, and 7 of 13 (54%) of MSI(+) cell lines examined (HCTl16, LOVO, LS44lN, LSl80, LSl74T, HCT8, and AN3CA). These mutations were somatic because the corresponding normal counterparts were wild type. With the exception of a mutation in the (A)₈ tract in KSl9 (gastric carcinoma), E75 (endometrial cancer), AC334 (colon cancer) and AC590 (colon cancer), all mutations targeted the (A)₉ tract. No RIZ mutations were found in 70 MSI(−) gastric carcinomas, indicating that these mutations are specific for MSI(+) tumors.

Among the 46 cases with RIZ mutations (19 gastric carcinomas, 6 endometrial carcinomas, 14 colorectal carcinomas, and 7 cell lines), eleven cases (KS15, KSl9, E4, E68, E75, E505, AC91, AC334, AC469, HCT-116 and AN3CA) were biallelic mutations. KS15, E68, E505, AC91, AC469 and HCT-116 showed homozygous/hemizygous mutations. KS19 and AC334 had a 1-bp deletion at both the (A)₈ and (A)₉ tracts in one allele and a 1-bp deletion at the (A)₉ tract in the other allele, whereas E4 and AN3CA showed a 1-bp deletion at the (A)₉ tract in one allele and a 2-bp deletion at the (A)₉ tract in the other allele. E75 had a 1-bp deletion at the (A)₈ tract in one allele, and a 2-bp deletion at the (A)₉ tract in the other allele.

To determine whether RIZ is also affected by genomic deletions in MSI (+) cancers, loss of heterozygosity (LOH) studies were performed on 25 cases with frameshift mutations for which the matched normal DNAs were available. The RIZ pro704 deletion polymorphism, a three-nucleotide deletion at codon Pro704 in exon 8 (Fang et al., Genes Chromosomes Cancer 28:269-275 (2000)), allowed detection of LOH in 2 of 12 informative tumors, KS07 and KS20, which had frameshift mutations in one allele. Therefore, eleven cancers (KS07, KS15, KSl9, KS20, E4, E68, E75, E505, AC91, AC334 and AC469) and two cell lines (HCT-116, AN3CA) had evidence of biallelic inactivation of RIZ.

All of the frame shift mutations in RIZ detected in this example are predicted to lead to the production of COOH-terminal domain truncated proteins. The deletion of one adenosine in the (A)₈ tract of RIZ produces a stop codon two residues from the tract and a mutant protein lacking the C-terminal 293 amino acids.

It has been demonstrated that the COOH-terminal domain of RIZl is a PR domain-binding motif, which may play a role in binding RIZl (RIZ oligomerization) (Huang et al., J. Biol. Chem. 273:15933-15940 (1998)). Thus, deletion of this COOH-terminal protein-binding interface is likely to seriously affect RlZl function.

These results show that RIZ frameshift mutations are common in sporadic MSI(+) cancers, including gastric, endometrial and colorectal carcinomas. Many of these mutations are biallelic or homozygous/hemizygous, which suggests that RIZ fits the Knudson two-hit model of tumor suppressor genes (Knudson, Proc. Natl. Acad. Sci. USA 68:820-823 (1971)). Given the characteristic low frequency of LOH in MSI(+) tumors, it is not surprising that LOH is not commonly found at the RIZ locus in these tumors.

In view of the recent report of a role of RIZ in estrogen-receptor signaling (Abbondanza et al., Proc. Natl. Acad. Sci. USA 97:3130-31135 (2000)), the finding of RIZ mutations in endometrial carcinomas is consistent with an important role in the hormone-dependent growth-control pathways in the endometrium.

### EXAMPLE III

### RIZ1 Expression Inhibits Growth of MSI(+) Tumors

This examples shows that RIZ1 expression inhibits growth of MSI(+) tumors and induces apoptosis of tumor cells in vivo.

To determine the efficacy of RIZl in gene therapy of MSI(+) tumors, the effect of expression of recombinant RIZl in inhibiting growth of established solid tumors was determined. As a control, the effect of recombinant p53 expression was also assessed.

The MSI(+) HCT116 colorectal cancer cell line (obtained from American Type Culture Collection), which carries homozygously mutated RIZl and wild type p53, was cultured in Dulbecco's modified Eagle's medium with 10% fetal calf serum.

Athymic female nu/nu (nude) mice were obtained from Jackson Laboratories-(Bar Harbor, Me.). HCT116 tumor cells (2×10⁶ cells in 100 μl PBS/mouse) were injected subcutaneously into nude mice. Tumors were allowed to grow in vivo for 6 days, at which time they reached an average size of 0.5 cm in diameter. Prior to therapy, the animals were randomized and regrouped by tumor size (5 to 11 mice per group). Mice bearing established HCT116 tumors received intratumoral and peritumoral injection of either PBS alone, or adenovirus suspension (8×10¹⁰ particles of virus per dose), on every other day for a total of 4 doses.

The adenovirus constructs lacking an insert (Adnull, gift of Prem Seth, National Cancer Institute, Maryland), or expressing either RIZl (AdRIZl) or p53 (Adp53) were prepared, amplified and titered in 293 cells, as described in He et al., Cancer Res. 58:4328-4244 (1998).

Tumor sizes were measured 2 to 3 times a week. Tumor volumes were calculated as a×b²×0.5, where a is the length and b is the width of the tumor in millimeters. Tumor volumes for different treatment groups on each day were compared by Student's t test.

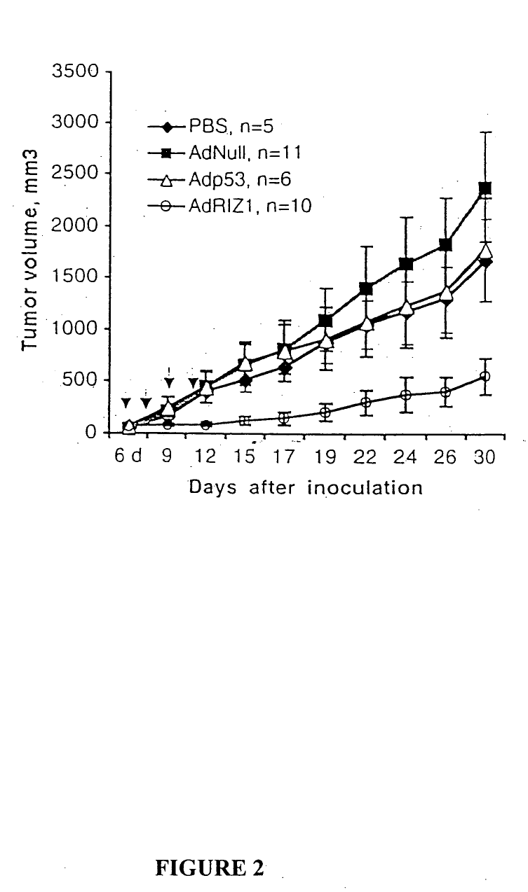

As shown in FIG. 2, in mice treated with either PBS alone, the Adnull virus, or the Adp53 virus, tumors continued to grow aggressively. In contrast, tumors in mice treated with AdRlZl virus grew significantly slower.

The effects of RIZ1 and p53 expression on tumor cell apoptosis in established tumors was also determined. HCT116 tumor cells were injected into nude mice, and the xenografts allowed to grow for one month. Virus suspension was injected intratumorally into the established tumors. At day 2 post-injection, tumors were excised, fixed in 10% buffered formalin and embedded in paraffin. Tissue sections were processed for immunohistochemical staining for the expression of RIZ1 or p53, or for the presence of fragmented DNA.

For RlZl immunostaining, antiserum 1637 was used at 1:400 dilution. For p53 staining, rabbit serum AB545 (Chemicon, Calif.) was used at 1:200 dilution. Secondary antibodies were peroxidase-labeled goat anti-rabbit IgG. For detection of apoptosis, Apoptag in situ apoptosis detection kits were used (Intergen, Purchase, N.Y.). Samples were assayed as per kit directions. Briefly, deparaffinized, rehydrated tissue sections were treated with proteases, incubated with TdT, and developed using an avidin-peroxidase kit and DAB (DAKO, San Francisco, Calif.). Slides were counterstained with hematoxylin.

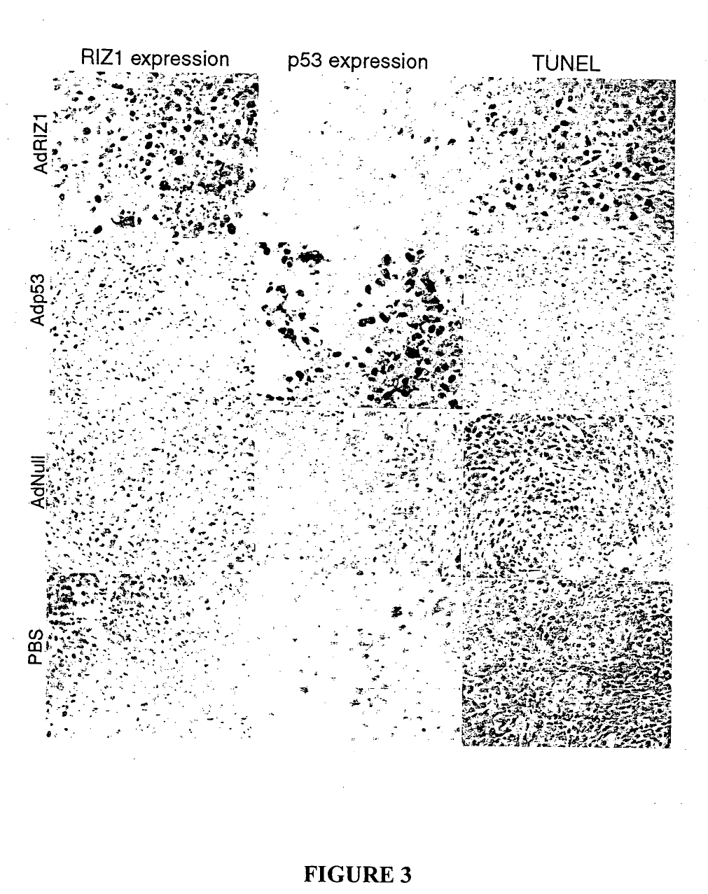

As shown in FIG. 3, the majority of tumor cells surrounding the injection sites showed strong RlZl or p53 nuclear staining in AdRIZl or Adp53 virus-injected tumors, respectively. In addition, tumors injected with AdRIZl, but not Adp53, Adnull, or buffer alone showed strong Apoptag staining, indicating that apoptosis occurred in RlZ1 expressing cells.

In summary, consistent with the in vitro observations described in Example I, above, the preclinical study described above demonstrates that treatment of MSI(+) tumors by introduction of an RIZ1 expression construct inhibits growth and induces apoptosis of the tumor

All journal article, reference and patent citations provided above, in parentheses or otherwise, whether previously stated or not, are incorporated herein by reference in their entirety.

Although the invention has been described with reference to the examples provided above, it should be understood that various modifications can be made without departing from the spirit of the invention. Accordingly, the invention is limited only by the claims.

1. A method of inhibiting growth of a microsatellite instability (MSI)-positive tumor, comprising introducing into said tumor a nucleic acid molecule encoding a RIZ1 polypeptide, and expressing said polypeptide in said tumor in an effective amount to inhibit growth of said tumor.

2. The method of claim 1, wherein said tumor contains cells having an abnormal number of adenosine nucleotides in a RIZ poly(A) tract.

3. The method of claim 2, wherein said poly(A) tract is the RIZ (A)₈ tract.

4. The method of claim 2, wherein said poly(A) tract is the RIZ (A)₉ tract.

5. The method of claim 2, wherein said abnormal number of adenosine residues is an increased number.

6. The method of claim 2, wherein said abnormal number of adenosine residues is a decreased number.

7. The method of claim 1, wherein expression of RIZ1 in said tumor induces apoptosis.

8. The method of claim 1, wherein said RIZ1 polypeptide comprises a PR domain amino acid sequence at least 95% identical to SEQ ID NO:5.

9. The method of claim 8, wherein said RIZ1 polypeptide comprises SEQ ID NO:5.

10. The method of claim 1, wherein said RIZ1 polypeptide comprises an amino acid sequence at least 80% identical to SEQ ID NO:4.

11. The method of claim 10, wherein said RIZ1 polypeptide comprises SEQ ID NO:4.

12. The method of claim 1, wherein said nucleic acid molecule is contained in a viral vector.

13. The method of claim 12, wherein said viral vector is an adenoviral vector.

14. The method of claim 1, wherein said nucleic acid molecule is administered intra- or peri-tumorally.

15. The method of claim 1, wherein said MSI-positive tumor is selected from the group consisting of a colorectal tumor, a gastric tumor and an endometrial tumor.

16. The method of claim 15, wherein said MSI-positive tumor is a hereditary nonpolyposis colon carcinoma.

17-23. (canceled)


SEQUENCE LISTING (15 sequences)

SEQ ID NO: 1 - DNA, 6171 bp, Rattus SP.
  CDS (157)...(5274)
gccaagatggcggcggcgcggccgcgggcgccagggcgacggcggcggctgaggctctgggctcgctgaagcgttggcacgtcgcgctctgggctcatgtaatcaaagaagtttctttgttgtgtgtatcttcacagaacacaacaggaattgaaaatgcatcagaacactgagtctgtggcagccactgagactctggctgaggtacctgaacatgtgcttcgaggacttccagaggaagtaagacttttcccatctgcagtcgacaagactcggattggtgtctgggctactaaaccaattttaaaagggaaaaagtttgggccatttgttggtgataagaagaagagatcccaggttaggaataatgtgtacatgtgggaggtctactacccaaatttggggtggatgtgcattgatgccaccgatccggagaagggcaactggctacgctatgtgaactgggcttgctcaggagaagagcagaatttatttccactggaaatcaacagagccatttactataaaaccttaaagccaatcgcgcctggcgaggagctcctggtctggtacaatggggaagacaaccctgagatagcagctgcgattgaggaagagcgagccagcgcccggagcaagcggagctccccgaagagccgcagagggaagaagaaatcacacgagaacaaaaacaaaggcatcagaacccaccccacacagctgaaggcaagtgagctggactctacctttgcaaacatgaggggctctgcagaaggtccaaaagaagaggatgagaggcctttggcttcggcacctgagcagccagcccctctgccggaggtggggaatcaagatgcagttccacaggtggccatccctctccctgcctgcgagccacagccagaggtagatgggaaacaagaagtcacagactgtgaggtcaatgatgtggaggaagaggagctggaagaggaagaggagctggaagaggaggaggaggaggagttgggagaagatggggtagaagaagcagacatgccgaatgaaagctctgcgaaagagccggagatccggtgtgaagaaaagccagaagacttattagaagagccacagagcatgtcgaatgaagctcgtgaagactctccagacgtgacccctcctccccacactcccagagctagagaggaggccaacggtgatgtacttgagacatttatgtttccgtgtcagcactgtgaaagaaaatttgcaacgaagcaggggctagagcgtcacatgcacatccacatttctaccatcaatcatgctttcaagtgcaagtactgtgggaaacggtttggcacacagattaacaggaggcggcatgaacggcgccacgaaacggggttgaagagaagacccagcatgactttacagtcctcagaggatccagatgatggcaagggggaaaatgttacttctaaagatgagtcaagtccacctcaactcgggcaagactgtttgatattgaactcagagaaaacctcacaggaagtactgaattcatcttttgtggaagaaaatggtgaagttaaagaacttcatccgtgcaaatactgcaaaaaggtatttggaactcacaccaatatgagacgacatcagcgtagagttcatgagcgccacctgattcccaaaggtgtcaggcgaaaaggaggacttctggaagagccacagccaccagcagagcaggctccaccctcccagaatgtctatgtcccaagcacagagccagaggaggaaggggaaacagatgacgtgtacatcatggacatttctagcaacatctctgaaaacctaaattactatattgacggtaagattcagaccaacagcagcactagtaactgtgatgtgattgagatggagtctaattctgcacacttgtatggcatagactgtctgctcactccagtgaccgtggagattactcagaacataaagagcactcaggtctctgtgacagatgatcttctcaaagactctcccagcagcacaaattgtgagtctaagaaacggaggactgccagtccacctgtgctccccaaaattaaaacggagacggagtctgattccacagcaccctcgtgttccttaagtctgcccctgagcatatccacagccgaggtggtgtccttccataaagagaagggcgtctatttgtcgtccaagctcaagcagcttcttcagacccaggacaagctgacccttcctgcagggttttcagcagctgagattcctaagttaggtcccgtgtgcgcgtctgctcctgcatccatgttgcccgtgacctctagtaggtttaagagacgcaccagctctccaccgagctctccacagcacagccctgcccttcgagacttcgggaaaccaaatgatgggaaagcagcatggacagacacagtcctgacttccaagaaacccaagttagaaagtcgtagtgactcaccagcatggagtttgtctgggagagatgaaagagaaaccggaagccctccttgctttgatgaatacaaaatatcaaaggaatgggcagccagttctactttcagcagtgtgtgcaaccaacagccattggatttatccagcggggtcaaacagaagtcagagggcacaggcaagactccagtcccatgggaatctgtattggatctcagtgtgcataaaaagccttgcgattctgaaggcaaggaattcaaagagaaccatttggcacagccagctgcaaagaagaaaaaaccaaccacctgtatgcttcaaaaggttcttctcaatgagtataatggtgttagcttacctacagaaaccacaccagaggtgaccaggagcccaagtccttgtaaatccccagatacacagccagatcctgaacttggtcctgactcaagttgctcagtccccactgctgagtctccacctgaagttgttggcccttcctcaccccctctccagacagcctccttatcctccggtcagctgcctcctctcttaacccccacagagccttcttcccctcccccctgccctcctgtgttaactgttgccactccaccacctcccctccttccaaccgtccctctctcccacccctcttctgatgcctcccctcagcagtgtccctctccgttctcaaacaccactgctcagtctcctcttcccattctctccccaacagtgtctccctctccctctcccattcctcctgtagagccacttatgtctgctgcttcccctggtcccccaacactttcttcctcctcctcttcttcctcttccttcccttcctcttcctgctcctccacctccccctccccaccccctctttcagcagtgtcatctgtggtttcctctggggacaacctggaggcatctctgcctgcagtaactttcaaacaggaggagtcagagagtgaaggtctgaaacccaaggaagaggccccacctgcagggggacagagtgtggtccaagaaacattcagcaaaaacttcatttgcaatgtctgtgaatcgccttttctttccattaaagacctaaccaaacatttatccgtccatgctgaagagtggcccttcaaatgtgagttttgtgtgcagctgtttaaggttaagactgatctatcagagcatcgatttctgcttcatggggttggaaatatctttgtgtgttctgtatgtaagaaagaatttgccttcttatgcaatctgcagcagcaccagcgtgatctccacccagatgaggtatgcacacaccacgagtttgaaagtgggaccctgaggccccagaacttcacagaccccagcaaggccaatgttgagcatatgccaagtttgccagaagagcctttagaaacttctagagaggaggagttaaatgattcctctgaagagctttacacgaccatcaaaataatggcttctggaataaagacgaaggatccagatgttcgacttggtctcaaccagcactacccgagctttaaacctcctccatttcagtaccaccatcgaaaccctatggggataggggtgacagccaccaacttcaccacccacaatattccacagactttcactactgccatccgctgcacaaagtgtgggaagggcgtcgacaatatgcctgagctgcataagcatatcttggcgtgtgcgtctgcaagtgacaagaagaggtacacccctaagaaaaacccagtgcccctgaaacaaactgtgcagcccaaaaatggagtggtggttctagacaactctgggaaaaatgccttcagacggatggggcagcccaagagactgagcttcaatgttgaactgggtaaaatgtctccaaacaagctcaagctgagtgcgctgaagaagaaaaaccagctggtgcagaaggccatccttcagaagaacagagccgcgaagcagaaggcggacctgagggatacttccgaggcgtcctcacacatctgcccgtactgtgacagggagttcacatacattggcagcctgaataagcatgccgccttcagctgtcctaaaaaacctctttctccttccaaaagaaaagtttcccattcgtctaagaaaggtggccatgcatcatcttctagcagtgacagaaacagcagctgccacccccggaggcggactgcagataccgagatcaagatgcagagcacgcaggcacccttgggcaagaccagagctcggagtacaggccccgcccaggcctcactgccctcctcgtccttcagatccagacagaatgtcaaatttgcagcttcagtgaaatccaaaaaagcaagctcttcatccttgaggaattccagtcccataagaatggccaaaattactcacgtcgagggcaaaaaacccaaagctgttgccaagagtcattctgctcagctctcaagcaaatcctcccgaggcctgcatgtcagagtgcagaagagcaaagctgtcatacagagcaagactgccctggccagtaagaggagaacagaccggttcatagtgaaatctagagagcgcagcgggggcccaatcacccgaagccttcagctggcagctgctgcggacctgagtgaaagcaggagagaggacagcagtgccaggcatgagctgaaggacttcagctacagtctccgcctggcatctcgatgcggctcatcaacagcctcttacatcaccagacaatgcagaaaggtcaaggccgccgcagcaactccgttccagggacccttcctcaaagagtaggcactctgtctgctccttaacagcacctgaagtgacctggaatcagtgaagccaaagggaccagcagtctgccctgcagagagcactgacctctcccagttgtgagagtgagagaacgagagagagagagagagagagagagagagagagagagagagagaatgagaatgtgtgtgtgtgtgtgctggtgcatgtgtgtggtcttcaagccaaggtcccagcctcaggagcaggaccttcccatttcccgtcatcctctggatgatccttggacgtggcccagaaccgtgctctgtggtgcagccatcctgcccgggaggggcatctccttctatgcaatttttttaaagagttccttggccctgctttgtgcttcttgagctgtccgtttgccaccactgggacttggatctggccctgaggggtggggaagagggcctatctaaggataacctttcagaggtcaagctccccttcatgccacccctcccccctgccctcaccgaccttttccccacactgtctctgggaatcaatagcagatagcatatagatccatcagggttgagcctgaacctcggccctagcactaggaaatcccccttttctccctaagcaactggagccgccagctttcaagtcatttcctcctttgaggttctagagtccgagagtctgctccgaagtctctcctgggaacccgggagccctcgcacccaggacgcagactctgtgcccattcttagacctgaggtagaagaagcagtgttttgggacgatagggtggaggcgtgcctactttgtctcctctggtgggacctcctacatcattggcatctgaaccttgcaagttcgctgcaaagagaagcaaaggaaaaaaaaaaaaaaaaaaaa

SEQ ID NO: 2 - protein, 1706 aa, Rattus SP.
MHQNTESVAATETLAEVPEHVLRGLPEEVRLFPSAVDKTRIGVWATKPILKGKKFGPFVGDKKKRSQVRNNVYMWEVYYPNLGWMCIDATDPEKGNWLRYVNWACSGEEQNLFPLEINRAIYYKTLKPIAPGEELLVWYNGEDNPEIAAAIEEERASARSKRSSPKSRRGKKKSHENKNKGIRTHPTQLKASELDSTFANMRGSAEGPKEEDERPLASAPEQPAPLPEVGNQDAVPQVAIPLPACEPQPEVDGKQEVTDCEVNDVEEEELEEEEELEEEEEEELGEDGVEEADMPNESSAKEPEIRCEEKPEDLLEEPQSMSNEAREDSPDVTPPPHTPRAREEANGDVLETFMFPCQHCERKFATKQGLERHMHIHISTINHAFKCKYCGKRFGTQINRRRHERRHETGLKRRPSMTLQSSEDPDDGKGENVTSKDESSPPQLGQDCLILNSEKTSQEVLNSSFVEENGEVKELHPCKYCKKVFGTHTNMRRHQRRVHERHLIPKGVRRKGGLLEEPQPPAEQAPPSQNVYVPSTEPEEEGETDDVYIMDISSNISENLNYYIDGKIQTNSSTSNCDVIEMESNSAHLYGIDCLLTPVTVEITQNIKSTQVSVTDDLLKDSPSSTNCESKKRRTASPPVLPKIKTETESDSTAPSCSLSLPLSISTAEVVSFHKEKGVYLSSKLKQLLQTQDKLTLPAGFSAAEIPKLGPVCASAPASMLPVTSSRFKRRTSSPPSSPQHSPALRDFGKPNDGKAAWTDTVLTSKKPKLESRSDSPAWSLSGRDERETGSPPCFDEYKISKEWAASSTFSSVCNQQPLDLSSGVKQKSEGTGKTPVPWESVLDLSVHKKPCDSEGKEFKENHLAQPAAKKKKPTTCMLQKVLLNEYNGVSLPTETTPEVTRSPSPCKSPDTQPDPELGPDSSCSVPTAESPPEVVGPSSPPLQTASLSSGQLPPLLTPTEPSSPPPCPPVLTVATPPPPLLPTVPLSHPSSDASPQQCPSPFSNTTAQSPLPILSPTVSPSPSPIPPVEPLMSAASPGPPTLSSSSSSSSSFPSSSCSSTSPSPPPLSAVSSVVSSGDNLEASLPAVTFKQEESESEGLKPKEEAPPAGGQSVVQETFSKNFICNVCESPFLSIKDLTKHLSVHAEEWPFKCEFCVQLFKVKTDLSEHRFLLHGVGNIFVCSVCKKEFAFLCNLQQHQRDLHPDEVCTHHEFESGTLRPQNFTDPSKANVEHMPSLPEEPLETSREEELNDSSEELYTTIKIMASGIKTKDPDVRLGLNQHYPSFKPPPFQYHHRNPMGIGVTATNFTTHNIPQTFTTAIRCTKCGKGVDNMPELHKHILACASASDKKRYTPKKNPVPLKQTVQPKNGVVVLDNSGKNAFRRMGQPKRLSFNVELGKMSPNKLKLSALKKKNQLVQKAILQKNRAAKQKADLRDTSEASSHICPYCDREFTYIGSLNKHAAFSCPKKPLSPSKRKVSHSSKKGGHASSSSSDRNSSCHPRRRTADTEIKMQSTQAPLGKTRARSTGPAQASLPSSSFRSRQNVKFAASVKSKKASSSSLRNSSPIRMAKITHVEGKKPKAVAKSHSAQLSSKSSRGLHVRVQKSKAVIQSKTALASKRRTDRFIVKSRERSGGPITRSLQLAAAADLSESRREDSSARHELKDFSYSLRLASRCGSSTASYITRQCRKVKAAAATPFQGPFLKE

SEQ ID NO: 3 - DNA, 5868 bp, Homo sapien
  CDS (121)...(5277)
ggaaaatttattcccactggaaatcaacagagccatttactataaaactttaaagggttcatgtaatcaaagaagtttcttgtgtgtgtgtatctttacagaacacaacaggaattgaaaatgaatcagaacactactgagcctgtggcggccaccgagaccctggctgaggtacccgaacatgtgctgcgaggacttccggaggaagtgaggcttttcccttctgctgttgacaagacccggattggtgtctgggccactaaaccaattttaaaagggaaaaaatttgggccatttgttggtgataagaaaaaaagatctcaggttaagaataatgtatacatgtgggaggtgtattacccaaatttgggatggatgtgcattgatgccactgatccagagaagggaaactggctgcgatatgtgaattgggcttgctcaggagaagagcaaaatttattcccactggaaatcaacagagccatttactataaaactttaaagccaatcgcgccgggcgaggagctcctggtctggtacaatggggaagacaaccctgagatagcagctgcgattgaggaagagcgagccagcgcccggagcaagcggagctcccccaagagccggaaagggaagaaaaaatcccaggaaaataaaaacaaaggaaacaaaatccaagacatacaactgaagacaagtgagccagatttcacctctgcaaatatgagagattctgcagaaggtcctaaagaagacgaagagaagccttcagcctcagcacttgagcagccggccaccctccaggaggtggccagtcaggaggtgcctccagaactagcaacccctgcccctgcctgggagccacagccagaaccagacgagcgattagaagcggcagcttgtgaggtgaatgatttgggggaagaggaggaggaggaagaggaggaggatgaagaagaagaagaagatgatgatgatgatgagttggaagacgagggggaagaagaagccagcatgccaaatgaaaattctgtgaaagagccagaaatacggtgtgatgagaagccagaagatttattagaggaaccaaaaacaacttcagaagaaactcttgaagactgctcagaggtaacacctgccatgcaaatccccagaactaaagaagaggccaatggtgatgtatttgaaacgtttatgtttccgtgtcaacattgtgaaaggaagtttacaaccaaacaggggcttgagcgtcacatgcatatccatatatccaccgtcaatcatgctttcaaatgcaagtactgtgggaaagcctttggcacacagattaaccggcggcgacatgagcggcgccatgaagcagggttaaagcggaaacccagccaaacactacagccgtcagaggatctggctgatggcaaagcatctggagaaaacgttgcttcaaaagatgattcgagtcctcccagtcttgggccagactgtctgatcatgaattcagagaaggcttcccaagacacaataaattcttctgtcgtagaagagaatggggaagttaaagaacttcatccgtgcaaatattgtaaaaaggtttttggaactcatactaatatgagacggcatcagcgtagagttcacgaacgtcatctgattcccaaaggtgtacggcgaaaaggaggccttgaagagccccagcctccagcagaacaggcccaggccacccagaacgtgtatgtaccaagcacagagccggaggaggaaggggaagcagatgatgtgtacatcatggacatttctagcaatatctctgaaaacttaaattactatattgatggtaaaattcaaactaataacaacactagtaactgtgatgtgattgagatggagtctgcttcggcagatttgtatggtataaattgtctgctcactccagttacagtggaaattactcaaaatataaagaccacacaggtccctgtaacagaagatcttcctaaagagcctttgggcagcacaaatagtgaggccaagaagcggagaactgcgagcccacctgcactgcccaaaattaaggccgaaacagactctgaccccatggtcccctcttgctctttaagtcttcctcttagcatatcaacaacagaggcagtgtctttccacaaagagaaaagtgtttatttgtcatcaaagctcaaacaacttcttcaaacccaagataaactaactcctcctgcagggatttcagcaactgaaatagctaaattaggtcctgtttgtgtgtctgctcctgcatcaatgttgcctgtgacctcaagtaggtttaagaggcggaccagctctcctcccagttctccacagcacagtcctgcccttcgagactttggaaagccaagtgatgggaaagcagcatggaccgatgccgggctgacttccaaaaaatccaaattagaaagtcacagcgactcaccagcatggagtttgtctgggagagatgagagagaaactgtgagccctccatgctttgatgaatataaaatgtctaaagagtggacagctagttctgcttttagcagtgtgtgcaaccagcagccactggatttatccagcggtgtcaaacagaaggctgagggtacaggcaagactccagtccagtgggaatctgtcttagatctcagtgtgcataaaaagcattgtagtgactctgaaggcaaggaattcaaagaaagtcattcagtgcagcctacgtgtagtgctgtaaagaaaaggaaaccaaccacctgcatgctgcagaaggttcttctcaatgaatataatggcatcgatttacctgtagaaaaccctgcagatgggaccaggagcccaagtccttgtaaatccctagaagctcagccagatcctgacctcggtccgggctctggtttccctgcccctactgttgagtccacacctgatgtttgtccttcatcacctgccctgcagacaccctccctttcatccggtcagctgcctcctctcttgatccccacagatccctcttcccctccaccctgtcccccggtattaactgttgccactccgccccctcccctccttcctaccgtacctcttccagccccctcttccagtgcatctccacacccatgcccctctccactctcaaatgccaccgcacagtccccacttccaattctgtccccaacagtgtccccctctccctctcccattcctcccgtggagcccctgatgtctgccgcctcacccgggcctccaacactttcttcttcctcctcttcatcttcctcctcctcttcgttttcttcttcatcttcctcctcttctccttctccacctcctctctccgcaatatcatctgttgtttcctctggtgataatctggaggcttctctccccatgatatctttcaaacaggaggaattagagaatgaaggtctgaaacccagggaagagccccagtctgctgctgaacaggatgttgttgttcaggaaacattcaacaaaaactttgtttgcaacgtctgtgaatcaccttttctttccattaaagatctaaccaaacatttatctattcatgctgaagaatggcccttcaaatgtgaattttgtgtgcagctttttaaggataaaacggacttgtcagaacatcgctttttgcttcatggagttgggaatatctttgtgtgttctgtttgtaaaaaagaatttgcttttttgtgcaatttgcagcagcaccagcgagatctccacccagataaggtgtgcacacatcacgagtttgaaagcgggactctgaggccccagaactttacagatcccagcaaggcccatgtagagcatatgcagagcttgccagaagatcctttagaaacttctaaagaagaagaggagttaaatgattcctctgaagagctttacacgactataaaaataatggcttctggaataaagacaaaagatccagatgttcgattgggcctcaatcagcattacccaagctttaaaccacctccatttcagtaccatcaccgtaaccccatggggattggtgtgacagccacaaatttcactacacacaatattccacagactttcactaccgccattcgctgcacaaagtgtggaaaaggtgtcgacaatatgccggagttgcacaaacatatcctggcttgtgcttctgcaagtgacaagaagaggtacacgcctaagaaaaacccagtaccattaaaacaaactgtgcaacccaaaaatggcgtggtggttttagataactctgggaaaaatgccttccgacgaatgggacagcccaaaaggcttaactttagtgttgagctcagcaaaatgtcgtcgaataagctcaaattaaatgcattgaagaaaaaaaatcagctagtacagaaagcaattcttcagaaaaacaaatctgcaaagcagaaggccgacttgaaaaatgcttgtgagtcatcctctcacatctgcccttactgtaatcgagagttcacttacattggaagcctgaataaacacgccgccttcagctgtcccaaaaaacccctttctcctcccaaaaaaaaagtttctcattcatctaagaaaggtggacactcatcacctgcaagtagtgacaaaaacagtaacagcaaccaccgcagacggacagcggatgcggagattaaaatgcaaagcatgcagactccgttgggcaagaccagagcccgcagctcaggccccacccaagtcccacttccctcctcatccttcaggtccaagcagaacgtcaagtttgcagcttcggtgaaatccaaaaaaccaagctcctcctctttaaggaactccagcccgataagaatggccaaaataactcatgttgaggggaaaaaacctaaagctgtggccaagaatcattctgctcagctttccagcaaaacatcgcggagcctgcacgtgagggtacagaaaagcaaagctgttttacaaagcaaatccaccttggcgagtaagaaaagaacagaccggttcaatataaaatctagagagcggagtggggggccagtcacccggagccttcagctggcagctgctgctgacttgagtgagaacaagagagaggacggcagcgccaagcaggagctgaaggacttcagctacagcctccgcttggcgtcccgatgctctccaccagccgcctcttacatcaccaggcagtataggaaggtcaaagctccggctgcagcccagttccagggaccattcttcaaagagtagacactctggctgctccctgacagcacctgaagtgacctggaatcagtgaagccaaagggactggcagtctgcctgcagggagtaccgacctatcccagttgtgtgaggctgcgagagaaagggagtgcatgtgcgcgcgtgcatgtgtgcgtgcgtgtgtgttcacgtgttctcgtgcgggccgtgagtggtcttcaaacgagggtcccgaaccccggggcggcaggaagggggccgactccacgctgtcctttgggatgatacttggatgtcagctcttgggaccgtgtctgcagcccagccttcctgttggggtggggcctctcctactatgcaatttttcaagagctccttgaccctgctttttgcttcttgagttgtcttttgccattatggggactttggtttgacccaggggtcagccctttaggaaggccttcaggaggaggccgagttccccttcagtaccacccctctctccccacctgcccgctcccggcaacatctctgggaatcaacagcatattgacacgttggagccgagcctgaacatgccctgaccccagcacatggaaaacccccttcctt

SEQ ID NO: 4 - protein, 1719 aa, Homo sapien
MNQNTTEPVAATETLAEVPEHVLRGLPEEVRLFPSAVDKTRIGVWATKPILKGKKFGPFVGDKKKRSQVKNNVYMWEVYYPNLGWMCIDATDPEKGNWLRYVNWACSGEEQNLFPLEINRAIYYKTLKPIAPGEELLVWYNGEDNPEIAAAIEEERASARSKRSSPKSRKGKKKSQENKNKGNKIQDIQLKTSEPDFTSANMRDSAEGPKEDEEKPSASALEQPATLQEVASQEVPPELATPAPAWEPQPEPDERLEAAACEVNDLGEEEEEEEEEDEEEEEDDDDDELEDEGEEEASMPNENSVKEPEIRCDEKPEDLLEEPKTTSEETLEDCSEVTPAMQIPRTKEEANGDVFETFMFPCQHCERKFTTKQGLERHMHIHISTVNHAFKCKYCGKAFGTQINRRRHERRHEAGLKRKPSQTLQPSEDLADGKASGENVASKDDSSPPSLGPDCLIMNSEKASQDTINSSVVEENGEVKELHPCKYCKKVFGTHTNMRRHQRRVHERHLIPKGVRRKGGLEEPQPPAEQAQATQNVYVPSTEPEEEGEADDVYIMDISSNISENLNYYIDGKIQTNNNTSNCDVIEMESASADLYGINCLLTPVTVEITQNIKTTQVPVTEDLPKEPLGSTNSEAKKRRTASPPALPKIKAETDSDPMVPSCSLSLPLSISTTEAVSFHKEKSVYLSSKLKQLLQTQDKLTPPAGISATEIAKLGPVCVSAPASMLPVTSSRFKRRTSSPPSSPQHSPALRDFGKPSDGKAAWTDAGLTSKKSKLESHSDSPAWSLSGRDERETVSPPCFDEYKMSKEWTASSAFSSVCNQQPLDLSSGVKQKAEGTGKTPVQWESVLDLSVHKKHCSDSEGKEFKESHSVQPTCSAVKKRKPTTCMLQKVLLNEYNGIDLPVENPADGTRSPSPCKSLEAQPDPDLGPGSGFPAPTVESTPDVCPSSPALQTPSLSSGQLPPLLIPTDPSSPPPCPPVLTVATPPPPLLPTVPLPAPSSSASPHPCPSPLSNATAQSPLPILSPTVSPSPSPIPPVEPLMSAASPGPPTLSSSSSSSSSSSSFSSSSSSSSPSPPPLSAISSVVSSGDNLEASLPMISFKQEELENEGLKPREEPQSAAEQDVVVQETFNKNFVCNVCESPFLSIKDLTKHLSIHAEEWPFKCEFCVQLFKDKTDLSEHRFLLHGVGNIFVCSVCKKEFAFLCNLQQHQRDLHPDKVCTHHEFESGTLRPQNFTDPSKAHVEHMQSLPEDPLETSKEEEELNDSSEELYTTIKIMASGIKTKDPDVRLGLNQHYPSFKPPPFQYHHRNPMGIGVTATNFTTHNIPQTFTTAIRCTKCGKGVDNMPELHKHILACASASDKKRYTPKKNPVPLKQTVQPKNGVVVLDNSGKNAFRRMGQPKRLNFSVELSKMSSNKLKLNALKKKNQLVQKAILQKNKSAKQKADLKNACESSSHICPYCNREFTYIGSLNKHAAFSCPKKPLSPPKKKVSHSSKKGGHSSPASSDKNSNSNHRRRTADAEIKMQSMQTPLGKTRARSSGPTQVPLPSSSFRSKQNVKFAASVKSKKPSSSSLRNSSPIRMAKITHVEGKKPKAVAKNHSAQLSSKTSRSLHVRVQKSKAVLQSKSTLASKKRTDRFNIKSRERSGGPVTRSLQLAAAADLSENKREDGSAKQELKDFSYSLRLASRCSPPAASYITRQYRKVKAPAAAQFQGPFFKE

SEQ ID NO: 5 - protein, 116 aa, Homo sapien
VDKTRIGVWATKPILKGKKFGPFVGDKKKRSQVKNNVYMWEVYYPNLGWMCIDATDPEKGNWLRYVNWACSGEEQNLFPLEINRAIYYKTLKPIAPGEELLVWYNGEDNPEIAAAI

SEQ ID NO: 6 - protein, 21 aa, Homo sapien
EWGPVTRSLQRSTKQELKDLQ

SEQ ID NO: 7 - protein, 22 aa, Homo sapien
GAEETAAAAAALGALRLGRRAR

SEQ ID NO: 8 - DNA, 391 bp, Mus musculus
  CDS (1)...(390)
gtgtactacccaaatttggggtggatgtgcattgatgccactgatccggagaagggcaactggctccgctatgtgaactgggcttgctcaggagaagaacagaatttatttccactggaaatcaacagagccatttactataaaaccttaaagccaatcgcgcctggcgaggagctcctggtctggtacaatggggaagacaaccccgagatagcagctgcgattgaggaagagcgagccagcgcccggagcaagcggagctccccgaagagccggagagggaagaagaaatcacaggagaataaaaacaaaggcatcagaacccaggctgcagcgcggaaggcgagcgagctggactccacctctgcaaacatgaggggctctgcagaag

SEQ ID NO: 9 - protein, 130 aa, Mus musculus
VYYPNLGWMCIDATDPEKGNWLRYVNWACSGEEQNLFPLEINRAIYYKTLKPIAPGEELLVWYNGEDNPEIAAAIEEERASARSKRSSPKSRRGKKKSQENKNKGIRTQAAARKASELDSTSANMRGSAE

SEQ ID NO: 10 - DNA, 18 bp, Artificial Sequence
  synthetic primer
tgtaaaacgacggccagt

SEQ ID NO: 11 - DNA, 18 bp, Artificial Sequence
  synthetic primer
caggaaacagctatgacc

SEQ ID NO: 12 - DNA, 20 bp, Artificial Sequence
  synthetic primer
gagctcagcaaaatgtcgtc

SEQ ID NO: 13 - DNA, 20 bp, Artificial Sequence
  synthetic primer
caagtcggccttctgctttg

SEQ ID NO: 14 - DNA, 20 bp, Artificial Sequence
  synthetic primer
tctcacatctgcccttactg

SEQ ID NO: 15 - DNA, 20 bp, Artificial Sequence
  synthetic primer
gtgatgagtgtccacctttc